(19) United States
(12) Patent Application Publication
(10) Pub. No.: US 2024/0238536 A1
(43) Pub. Date: Jul. 18, 2024

(54) NEBULIZING MODULE

(71) Applicant: HCMed Innovations Co., LTD., Taipei City (TW)

(72) Inventor: CHANG-HSIEH YAO, Taipei City (TW)

(21) Appl. No.: 18/355,389

(22) Filed: Jul. 19, 2023

(30) Foreign Application Priority Data
Jan. 18, 2023 (CN) 202310073574.0

(51) Int. Cl. A61M 11/00

(52) U.S. Cl. CPC A61M 11/005; A61M 2207/10

(57) ABSTRACT

A nebulizing module including a base, an auxiliary fixing element, a nebulizing unit, and an adhesive is provided. The base includes a supporting portion, and the supporting portion has a first opening and a first engaging surface surrounding the first opening. The auxiliary fixing element is disposed on the supporting portion. The auxiliary fixing element has a second opening and a second engaging surface surrounding the second opening. The second engaging surface faces the first engaging surface. The nebulizing unit is disposed between the supporting portion and the auxiliary fixing element. The adhesive is disposed in gaps formed between the first engaging surface, the second engaging surface, and the nebulizing unit.

### CROSS-REFERENCE TO RELATED PATENT APPLICATION

This application claims the benefit of priority to China Patent application Ser. No. 202310073574.0, filed on Jan. 18, 2023 in People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a nebulizing module, and more particularly to a nebulizing module having improved structural strength.

### BACKGROUND OF THE DISCLOSURE

A nebulizer is used to atomize a medicinal liquid through a nebulizing assembly therein, so as to form an aerosol and then spray the aerosol outward. A nebulizing unit inside the nebulizing assembly can generate vibration through the electrical conduction of piezoelectric elements, thereby converting the medicinal liquid into the aerosol having tiny droplets. However, when the nebulizing unit is disposed on a supporting member for vibration and is connected with the supporting member by way of welding, the long-term high-frequency vibration causes an engaging surface between the nebulizing unit and the supporting member to peel away, such that gaps are formed therebetween. In a more severe scenario, the nebulizing unit may detach at the engaging surface, such that the nebulizing assembly becomes ineffective.

On the other hand, the current nebulizing unit and the supporting member can also be glued together by using an adhesive. However, the size of current nebulizing unit is relatively small, which causes an adhesion area of the adhesive to be limited, and an adhesion strength between the nebulizing unit and the supporting member to be insufficient.

In other words, after the nebulizing assembly vibrates for a long period of time, the engaging surface between the nebulizing unit and the supporting member can easily detach such that gaps are formed, so as to cause water leakage or the nebulizing unit to fall off, thereby affecting the service life of the nebulizing assembly.

Therefore, how to improve structural strength of the nebulizing assembly through the improvement of the structure design, so as to overcome the above-mentioned inadequacy, has become an important issue to be addressed in the art.

### SUMMARY OF THE DISCLOSURE

In response to the above-referenced technical inadequacies, the present disclosure provides a nebulizing module capable of addressing an issue of a nebulizing unit being easily detached due to vibrations in related art.

In one aspect, the present disclosure provides a nebulizing module. The nebulizing module includes a base, an auxiliary fixing element, a nebulizing unit, and an adhesive. The base includes a supporting portion. The supporting portion has a first opening and a first engaging surface surrounding the first opening. The auxiliary fixing element is disposed on the supporting portion. The auxiliary fixing element has a second opening and a second engaging surface surrounding the second opening. The second engaging surface faces the first engaging surface. The nebulizing unit is disposed between the supporting portion and the auxiliary fixing element. The adhesive is disposed in gaps formed between the first engaging surface, the second engaging surface, and the nebulizing unit.

Therefore, in the nebulizing module provided by the present disclosure, by the auxiliary fixing element, the nebulizing unit, and the adhesive being fixed to each other, the nebulizing unit can be stably disposed on the base, so that the structural strength of the nebulizing module can be enhanced to satisfy requirements of resisting long-term vibrations.

Furthermore, in the nebulizing module provided by the present disclosure, by virtue of “the first engaging surface including a plurality of first grooves” and “the adhesive being filled into the plurality of first grooves,” the adhesion area between the adhesive and the base can be increased, so as to strengthen the adhesion between the nebulizing unit and the supporting area, thereby enhancing the overall structural strength of the nebulizing module.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a nebulizing module according to the present disclosure;

FIG. 2 is a schematic exploded view of the nebulizing module according to the present disclosure;

FIG. 3 is a schematic cross-sectional view taken along line III-III of FIG. 1;

FIG. 4 is a schematic view of a plurality of first grooves of a first engaging surface according to a first embodiment of the present disclosure;

FIG. 5 is a schematic view of a plurality of second grooves of a second engaging surface according to the first embodiment of the present disclosure;

FIG. 6 is a schematic view of a plurality of first grooves of a first engaging surface according to a second embodiment of the present disclosure;

FIG. 7 is a schematic view of a plurality of second grooves of a second engaging surface according to the second embodiment of the present disclosure;

FIG. 8 is a partial schematic cross-sectional view of a plurality of grooves being formed inside the nebulizing module according to the present disclosure;

FIG. 9 is a partial schematic exploded view of the nebulizing module and a piezoelectric element according to the present disclosure; and

FIG. 10 is a partial schematic cross-sectional view of the nebulizing module and the piezoelectric element according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### [Embodiments]

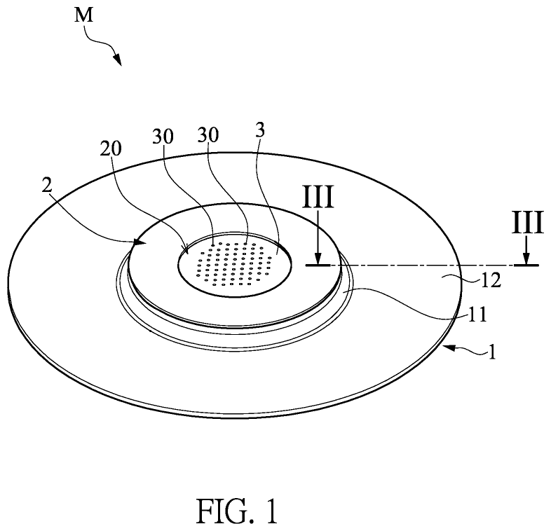

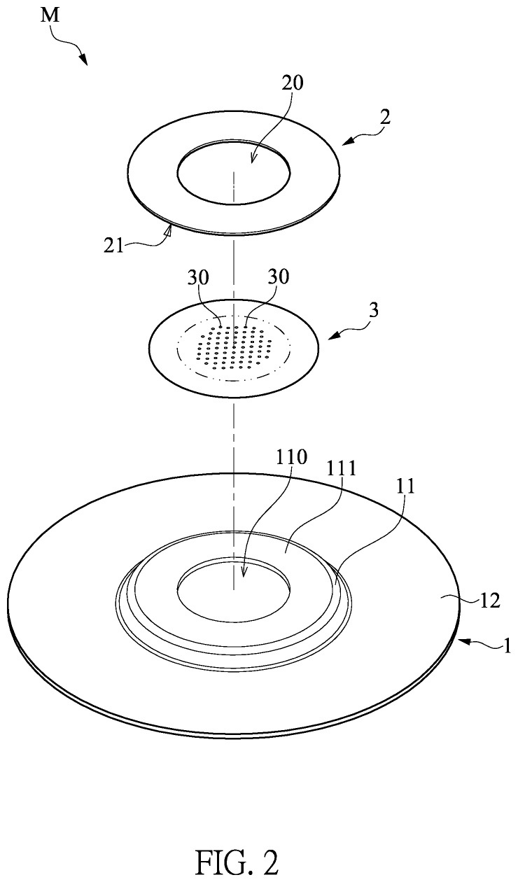

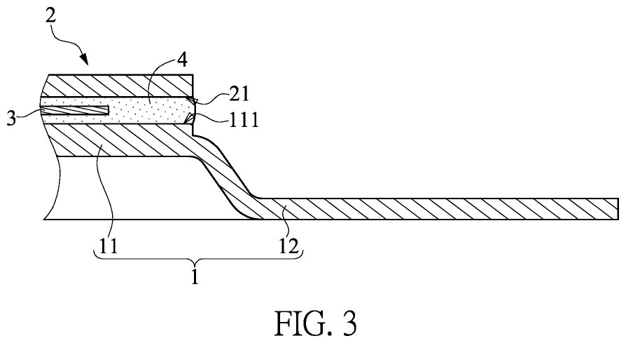

Referring to FIG. 1 and FIG. 2, FIG. 1 is a schematic view of a nebulizing module according to the present disclosure, and FIG. 2 is a schematic exploded view of the nebulizing module according to the present disclosure. The present disclosure provides a nebulizing module M, which includes a base 1, an auxiliary fixing element 2, and a nebulizing unit 3. The base 1 includes a supporting portion 11. The supporting portion 11 has a first opening 110 and a first engaging surface 111 surrounding the first opening 110. The auxiliary fixing element 2 is disposed on the supporting portion 11. The auxiliary fixing element 2 has a second opening 20 and a second engaging surface 21 surrounding the second opening 20. As shown in FIG. 2 and FIG. 3, the second engaging surface 21 faces the first engaging surface 111. The nebulizing unit 3 is disposed between the supporting portion 11 and the auxiliary fixing element 2. Specifically, the auxiliary fixing element 2 and the nebulizing unit 3 are stacked above the first engaging surface 111 of the supporting portion 11.

As shown in FIG. 3, FIG. 3 is a schematic cross-sectional view taken along line III-III of FIG. 1. The nebulizing module M further includes an adhesive 4. The adhesive 4 is disposed in gaps formed between the first engaging surface 111, the second engaging surface 21, and the nebulizing unit 3, such that the adhesive 4 can be glued between the base 1 and the auxiliary fixing element 2. For example, in the embodiment shown in FIG. 3, when the adhesive 4 is disposed in the gaps formed between the first engaging surface 111, the second engaging surface 21, and the nebulizing unit 3, the nebulizing unit 3 is not in contact with the first engaging surface 111 and the second engaging surface 21. However, in other embodiments, a part of the nebulizing unit 3 facing the first engaging surface 111 can be in contact with the first engaging surface 111, and a part of the nebulizing unit 3 facing the second engaging surface 21 can be in contact with the second engaging surface 21. A material of which the nebulizing unit 3 of the present disclosure is made is palladium-nickel alloy that has better chemical resistance and mechanical strength, and a material of which the adhesive 4 is made is epoxy resin, but the present disclosure is not limited thereto.

The adhesion area of the adhesive 4 between the supporting portion 11 of the base 1 and the auxiliary fixing element 2 can be increased through the configuration of the auxiliary fixing element 2, such that the nebulizing unit 3 can be stably fixed on the supporting portion 11 of the base 1. Accordingly, when the nebulizing module M vibrates at high frequency, the overall structural strength of the nebulizing module M can be effectively ensured.

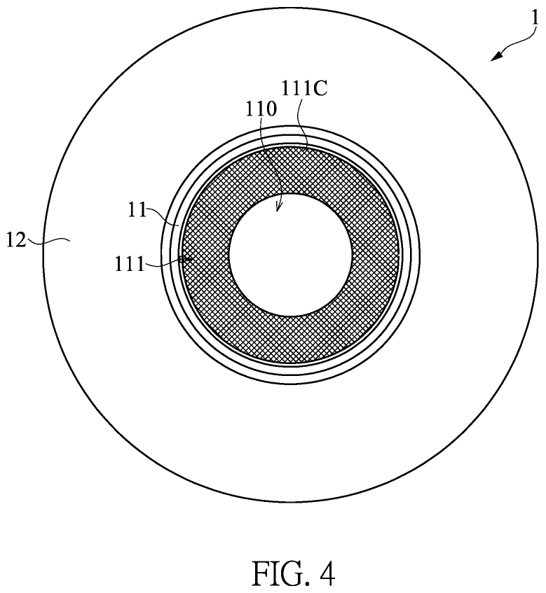

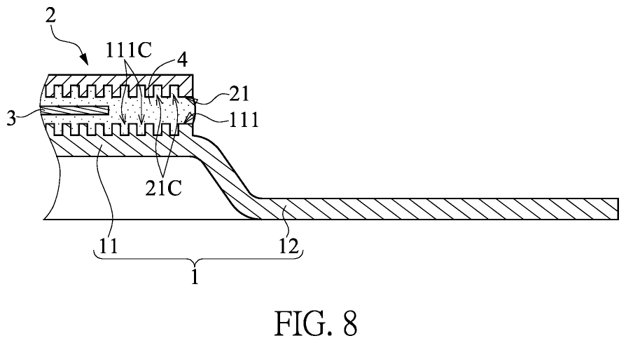

Referring to FIG. 4 and FIG. 8, FIG. 4 is a schematic view of a plurality of first grooves of a first engaging surface according to a first embodiment of the present disclosure, and FIG. 8 is a partial schematic cross-sectional view of a plurality of grooves being formed inside the nebulizing module according to the present disclosure. A surface of the first engaging surface 111 of the supporting portion 11 can be engraved by using a laser so that a plurality of first grooves 111C are formed thereon. Therefore, when the adhesive 4 is disposed on the first engaging surface 111, the adhesive 4 can be filled into the plurality of first grooves 111C. Furthermore, when the adhesive 4 is disposed in the gaps formed between the first engaging surface 111, the second engaging surface 21, and the nebulizing unit 3, the adhesive 4 can be filled into the plurality of first grooves 111C so as to increase the adhesion area between the adhesive 4 and the base 1.

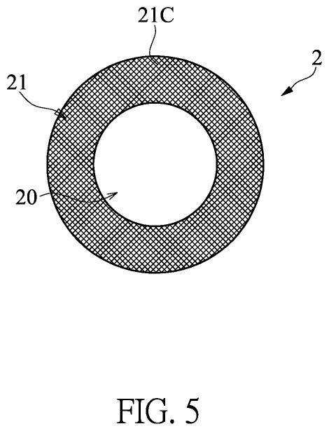

Referring to FIG. 4, FIG. 5, and FIG. 8, FIG. 5 is a schematic view of a plurality of second grooves of a second engaging surface according to the first embodiment of the present disclosure. In addition to the first engaging surface 111 of the supporting portion 11 having the plurality of first grooves 111C, the second engaging surface 21 of the auxiliary fixing element 2 can also be engraved by using the laser to have a plurality of second grooves 21C formed thereon. Similarly, when the adhesive 4 is disposed in the gaps formed between the first engaging surface 111, the second engaging surface 21, and the nebulizing unit 3, the adhesive 4 is filled into the plurality of second grooves 21C, thereby increasing the adhesion area between the adhesive 4 and the second engaging surface 21.

In a preferred embodiment, a width of each of the first grooves 111C and each of the second grooves 21C ranges between 80 μm and 120 μm, and a depth thereof ranges between 10 μm and 20 μm, but the present disclosure is not limited thereto.

Accordingly, an area of an orthogonal projection of the nebulizing unit 3 projected onto the first engaging surface 111 is smaller than an area of the first engaging surface 111. Moreover, the area of the orthogonal projection of the nebulizing unit 3 projected onto the first engaging surface 111 overlaps with areas of the first grooves 111C that are distributed on the first engaging surface 111, and the area of the orthogonal projection of the nebulizing unit 3 projected onto the first engaging surface 111 is smaller than the areas of the first grooves 111C that are distributed on the first engaging surface 111. Similarly, an area of an orthogonal projection of the nebulizing unit 3 projected onto the second engaging surface 21 is smaller than an area of the second engaging surface 21. Moreover, the area of the orthogonal projection of the nebulizing unit 3 projected onto the second engaging surface 21 overlaps with areas of the second grooves 21C that are distributed on the second engaging surface 21, and the area of the orthogonal projection of the nebulizing unit 3 projected onto the second engaging surface 21 is smaller than the areas of the second grooves 21C that are distributed on the second engaging surface 21.

Furthermore, the adhesive 4 is filled into the gaps (including space in the plurality of first grooves 111C) between the first engaging surface 111 and the nebulizing unit 3. At this time, the adhesive 4 is filled into the gaps (including space in the plurality of second grooves 21C) between the second engaging surface 21 and the nebulizing unit 3, and is filled into the gaps (including the space in the plurality of first grooves 111C and the second grooves 21C) between the first engaging surface 111 and the second engaging surface 21. In other words, the adhesion area between the adhesive 4, the base 1, and the auxiliary fixing element 2 can be greatly increased through the aforementioned structural design, and the gaps located between the first engaging surface 111, the second engaging surface 21, and the nebulizing unit 3 can be fully filled by the adhesive 4.

In one preferred embodiment, the first engaging surface 111 and the second engaging surface 21 can be rough surfaces treated by surface roughening. The adhesion force of the adhesive 4 between the base 1, the auxiliary fixing element 2, and the nebulizing unit 3 can be increased through the design of the grooves (i.e., the first grooves 111C and the second grooves 21C) and the rough surfaces, thereby enhancing the structural strength of the nebulizing module M.

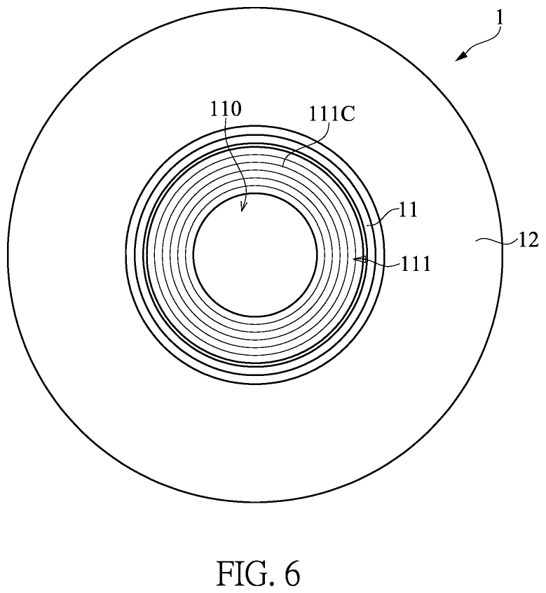

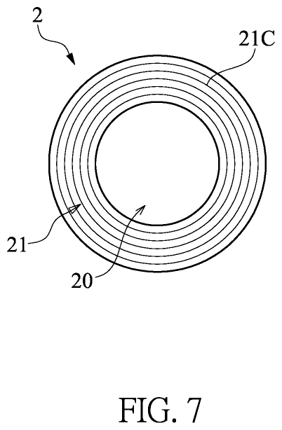

In the embodiment that is shown in FIGS. 4 and 5, the plurality of first grooves 111C and the plurality of second grooves 21C are in a shape of grids. However, the present disclosure is not limited thereto. Referring to FIG. 6 and FIG. 7, FIG. 6 is a schematic view of a plurality of first grooves of a first engaging surface according to a second embodiment of the present disclosure, and FIG. 7 is a schematic view of a plurality of second grooves of a second engaging surface according to the second embodiment of the present disclosure. In the embodiment shown in FIGS. 6 and 7, the plurality of first grooves 111C and the plurality of second grooves 21C are in a shape of concentric circles. It should be noted that, the aforementioned description for shapes of the first grooves 111C and the second grooves 21C is only an example, and is not meant to limit the scope of the present disclosure. For example, in other embodiments, the plurality of first grooves 111C are in a shape of grids, and the plurality of second grooves 21C are in a shape of concentric circles. Alternatively, the plurality of first grooves 111C are in a shape of concentric circles, and the plurality of second grooves 21C are in a shape of grids.

Reference is further made to FIGS. 1 and 2. The base 1 further includes a peripheral region 12, the peripheral region 12 surrounds the supporting portion 11, and the supporting portion 11 protrudes relative to the peripheral region 12. It can be seen from the appearance of the base 1 that the supporting portion 11 of the base 1 is a protruded platform. In addition, the nebulizing unit 3 includes a plurality of through holes 30. When the base 1, the auxiliary fixing element 2, and the nebulizing unit 3 are assembled to form the nebulizing module M, the plurality of through holes 30, the first opening 110, and the second opening 20 are in communication with each other.

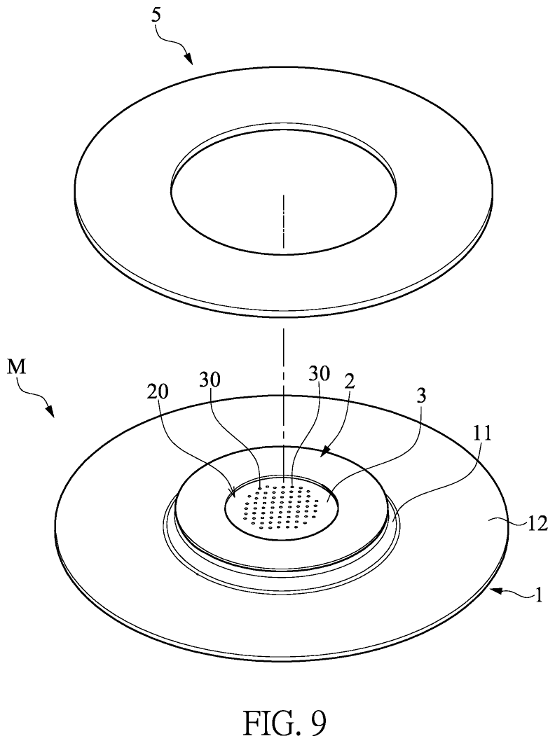

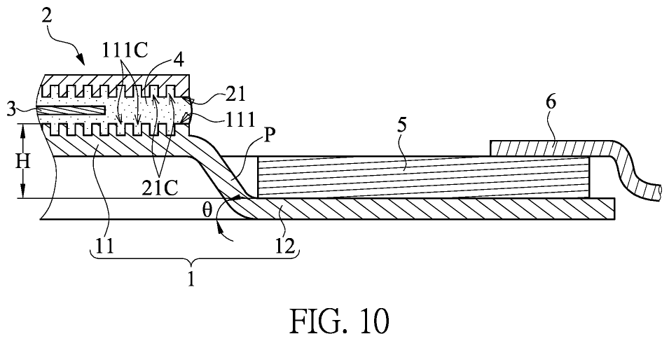

Referring to FIG. 9 and FIG. 10, FIG. 9 is a partial schematic exploded view of the nebulizing module and a piezoelectric element according to the present disclosure, and FIG. 10 is a partial schematic cross-sectional view of the nebulizing module and the piezoelectric element according to the present disclosure. The nebulizing module M further includes a piezoelectric element 5. The piezoelectric element 5 is a ring-shaped structure that is disposed on and in contact with the peripheral region 12. As shown in FIG. 10, the piezoelectric element 5 can be connected to a conductive wire 6 to receive an electronic voltage to vibrate, so as to drive the nebulizing unit 3 to vibrate together. Therefore, when the medicinal liquid passes through the plurality of through holes 30 of the vibrating nebulizing unit 3, the medicinal liquid can form an aerosol that is sprayed to the external environment. The present disclosure is not limited to a quantity of the conductive wire 6, and only one conductive wire 6 is shown in FIG. 10. In addition, by the protrusion design of the supporting portion 11, the vibration effect transmitted to the nebulizing unit 3 above the supporting portion 11 can be amplified when the piezoelectric element 5 receives the electronic voltage.

Regarding the protrusion design of the supporting portion 11, for example, the supporting portion 11 and the peripheral region 12 have a height difference H therebetween, and the height difference H ranges between 550 μm and 650 μm so as to amplify the amplitude of vibration transmitted from the piezoelectric element 5 to the nebulizing unit 3 when the piezoelectric element 5 receives the electronic voltage. Alternatively, the supporting portion 11 and the peripheral region 12 have a supporting inclined plane P therebetween, the supporting inclined plane P is inclined relative to a horizontal plane by an inclined angle θ, and the inclined angle θ is between 35 degrees and 45 degrees so as to optimize the efficiency of the vibration transmitted from the piezoelectric element 5 to the nebulizing unit 3 when the piezoelectric element 5 receives the electronic voltage.

### [Beneficial Effects of the Embodiments]

In the related art, engaging surfaces of the components of the nebulizing module are smooth surfaces, so that the adhesion force of the adhesive is insufficient. Therefore, gaps are easily formed between the components and the adhesive, and peeling of the components occurs after the nebulizing module vibrates for a long duration of time. Therefore, in the nebulizing module M provided by the present disclosure, by the auxiliary fixing element, the nebulizing unit, and the adhesive being fixed to each other, the nebulizing unit 3 can be stably disposed on the supporting portion 11 of the base 1, such that the structural strength of the nebulizing module M can be enhanced. On the other hand, the plurality of first grooves 111C can be formed on the first engaging surface 111 of the supporting portion 11 by laser engraving, and the plurality of second grooves 21C can be formed on the second engaging surface 21 of the auxiliary fixing element 2 by laser engraving, such that the adhesion area between the adhesive 4, the base 1, and the auxiliary fixing element 2 can be increased, thereby enhancing the structural strength of the nebulizing module M. In addition, the surface roughness of the first engaging surface 111 and the second engaging surface 21 can be increased by surface roughening, such that the adhesion area between the adhesive 4, the base 1, and the auxiliary fixing element 2 can be further increased. In addition, the gaps located between the first engaging surface 111, the second engaging surface 21, and the nebulizing unit 3 can be fully filled by the adhesive 4, so that the adhesion force of the adhesive 4 between the base 1, the auxiliary fixing element 2, and the nebulizing unit 3 can be increased, thereby enhancing the structural strength of the nebulizing module M and preventing the components from peeling off.

Furthermore, in the nebulizing module M provided by the present disclosure, by structural design of the area of the orthogonal projection of the nebulizing unit 3 projected onto the first engaging surface 111 being smaller than the area of the first engaging surface 111 and overlapping with areas of the first grooves 111C distributed on the first engaging surface 111, and the area of the orthogonal projection of the nebulizing unit 3 projected onto the second engaging surface 21 being smaller than the area of the second engaging surface 21 and overlapping with areas of the second grooves 21C distributed on the second engaging surface 21, parts of the nebulizing unit 3 which is used to engage the base 1 and the auxiliary fixing element 2 is covered by the first engaging surface 111 and the second engaging surface 21, such that the adhesive 4 can fully cover the parts of the nebulizing unit 3 and strengthen the adhesion strength between the base 1, the auxiliary fixing element 2, and the nebulizing unit 3, thereby improving the structural stability of the nebulizing module M.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A nebulizing module, comprising:
a base including a supporting portion, wherein the supporting portion has a first opening and a first engaging surface surrounding the first opening;
an auxiliary fixing element disposed on the supporting portion, wherein the auxiliary fixing element has a second opening and a second engaging surface surrounding the second opening, and the second engaging surface faces the first engaging surface;
a nebulizing unit disposed between the supporting portion and the auxiliary fixing element; and
an adhesive disposed in gaps formed between the first engaging surface, the second engaging surface, and the nebulizing unit.

2. The nebulizing module according to claim 1, wherein an area of an orthogonal projection of the nebulizing unit projected onto the first engaging surface is smaller than an area of the first engaging surface.

3. The nebulizing module according to claim 1, wherein the first engaging surface includes a plurality of first grooves, and the adhesive is filled into the plurality of first grooves.

4. The nebulizing module according to claim 3, wherein an area of an orthogonal projection of the nebulizing unit projected onto the first engaging surface overlaps with areas of the first grooves that are distributed on the first engaging surface, and the area of the orthogonal projection of the nebulizing unit projected onto the first engaging surface is smaller than the areas of the first grooves that are distributed on the first engaging surface.

5. The nebulizing module according to claim 3, wherein the plurality of first grooves are in a shape of grids or concentric circles.

6. The nebulizing module according to claim 3, wherein the second engaging surface includes a plurality of second grooves, and the adhesive is filled into the plurality of second grooves.

7. The nebulizing module according to claim 6, wherein an area of an orthogonal projection of the nebulizing unit projected onto the second engaging surface is smaller than an area of the second engaging surface.

8. The nebulizing module according to claim 7, wherein the area of the orthogonal projection of the nebulizing unit projected onto the second engaging surface overlaps with areas of the second grooves that are distributed on the second engaging surface, and the area of the orthogonal projection of the nebulizing unit projected onto the second engaging surface is smaller than the areas of the second grooves that are distributed on the second engaging surface.

9. The nebulizing module according to claim 6, wherein the plurality of second grooves are in a shape of grids or concentric circles.

10. The nebulizing module according to claim 1, wherein the nebulizing unit has a plurality of through holes, and the plurality of through holes, the first opening, and the second opening are in communication with each other.

11. The nebulizing module according to claim 10, wherein the base further includes a peripheral region, the peripheral region surrounds the supporting portion, and the supporting portion protrudes relative to the peripheral region.

12. The nebulizing module according to claim 11, wherein the supporting portion and the peripheral region have a height difference therebetween, and the height difference ranges between 550 μm and 650 μm.

13. The nebulizing module according to claim 11, wherein the supporting portion and the peripheral region have a supporting inclined plane therebetween, the supporting inclined plane is inclined relative to a horizontal plane by an inclined angle, and the inclined angle ranges between 35 degrees and 45 degrees.

14. The nebulizing module according to claim 11, further comprising a piezoelectric element, wherein the piezoelectric element is disposed on a side of the peripheral area facing the nebulizing unit.

15. The nebulizing module according to claim 1, wherein the first engaging surface and the second engaging surface are rough surfaces treated by surface roughening.
